(19) United States
(12) Patent Application Publication
(10) Pub. No.: US 2003/0022200 A1
(43) Pub. Date: Jan. 30, 2003

(54) Systems for analysis of biological materials

(76) Inventors: Henrik Vissing, Virum (DK); Mogens Havsteen Jakobsen, Vanlose (DK); Jens Godsk Kolberg, Espergaerde (DK)

Correspondence Address: EDWARDS & ANGELL, LLP., P.O. BOX 9169, BOSTON, MA, 02209, US

(21) Appl. No.: 10/105,949

(22) Filed: Mar. 25, 2002

Related U.S. Application Data
(60) Provisional application No. 60/278,592, filed on Mar. 25, 2001.

(51) Int. Cl. C12Q 1/68

(52) U.S. Cl. 435/6

(57) ABSTRACT

The present invention features methods for self-learning, self-engineering microarrays for the analysis of complex mixtures of nucleic acids and systems and apparatuses embodying such methods. In more particular aspects, the present invention features methods and systems that include inputting data relating to one or more biomolecules into a database; identifying at least one of a plurality of groupings for data to be obtained (mined) from the universal database; inputting at least one of the groupings into a neural network program, which can analyze the data and generate new design rules for predicting biological data; and adapting a software program including initial design rules to the new design rules.

The present application claims the benefit of U.S. provisional application number 60/278,592, filed Mar. 25, 2001, which is incorporated herein by reference in its entirety.

### BACKGROUND OF THE INVENTION

1. Field of the Invention.

The present invention features methods for self-learning, self-engineering micro arrays for the analysis of complex mixtures of nucleic acids and systems and apparatuses embodying such methods. In more particular aspects, the present invention features methods and systems that include inputting data relating to one or more biomolecules into a database; identifying at least one of a plurality of groupings for data to be obtained from the universal database; inputting at least one of the groupings into a neural network program, which can analyze the data and generate new design rules for predicting biological data; and adapting a software program including initial design rules to the new design rules.

2. Background.

The development of bio-array technologies promises to revolutionize the way biological research is carried out. Bio-arrays, wherein a library of biomolecules is immobilized on a small slide or chip, allow hundreds or thousands of assays to be carried out simultaneously on a miniaturized scale. This permits researchers to quickly gain large amounts of information from a single sample. In many cases, bio-array type of analysis would be not be possible using traditional biological techniques due to the rarity of the sample being tested and the time and expense necessary to carry out such large scale analysis.

There are two fundamentally different approaches to the manufacture of bio-arrays 1) “in situ synthesis” and 2) “micro spotting”. The in situ synthesis approach involves monomer-by-monomer synthesis directly on the substrate carrier. This approach has some inherent drawbacks as the synthesis of oligomers includes many chemical steps which never provide a 100% yield. Thus, bio-arrays produced via the in situ synthesis strategy generally contain truncated sequences leading to differences in the composition from array to array.

The micro spotting approach involves dispensing biomolecules onto the substrate carrier followed by immobilization of the molecules onto the surface. This approach offers the advantage that materials can be obtained from natural sources, or synthesized on standard synthesizers, purified and characterized prior to construction of the array. Thus, bio-arrays produced by the micro spotting approach generally are more reproducible and of higher quality than bio-arrays produced by the in situ synthesis approach.

Although bio-arrays are a powerful research tool, they do have a number of shortcomings in addition to that described above. For example, bio-arrays tend to be expensive to produce due to difficulties involved in reproducibly manufacturing high quality arrays. Also bio-array techniques cannot always provide the sensitivity necessary to perform a desired experiment. Therefore, it would be desirable to provide an improved platform for the production of arrays which results in less expensive, more reproducible and more sensitive bio-array.

Once this vast amount of data has been accumulated, there is a need for computer systems and programs for collection and analysis of this vast amount of information. Devices and computer systems have been developed for collecting information about gene expression or expressed sequence tags (EST) in large numbers of samples. For example, PCT application WO92/10588, incorporated herein by reference for all purposes, describes techniques for sequence checking nucleic acids and other materials. Probes for performing these operations may be formed in arrays are reported in U.S. Pat. Nos. 5,143,854 and 5,571,639.

Certain computer-aided techniques for gene expression monitoring using such arrays of probes have been developed as disclosed in EPO Pub. No. 0848067 and PCT publication No. WO 97/10365, the contents of which are herein incorporated by reference. See also U.S. Pat. Nos. 5,556,749; 6,242,180; and 6,251,588 and U.S. patent application Publication 2002/0028923. Many diseases are characterized by differences in the degree that various genes are expressed either through changes in the copy number of the genetic DNA or through changes in levels of transcription (e.g., through control of initiation, provision of RNA precursors, RNA processing, etc.) of particular genes. For example, losses and gains of genetic material play an important role in malignant transformation and progression. Furthermore, changes in the expression (transcription) levels of particular genes (e.g., oncogenes or tumor suppressors), serve as signposts for the presence and progression of various cancers.

Information on expression of genes or expressed sequence tags may be collected on a large scale in many ways, including the probe array techniques described above. One of the objectives in collecting this information is the identification of genes or ESTs whose expression is of particular importance. Collecting vast amounts of expression data from large numbers of samples including many tissue types is useful in answering these questions. However, in order to derive full benefit from the investment made in collecting and storing expression data, techniques enabling one to efficiently mine the data to find items of particular relevance are highly desirable.

It thus would be desirable to provide new methods and systems for manipulating data obtained from biological materials.

### SUMMARY OF THE INVENTION

The present invention features methods and techniques for organizing expression or concentration of information relating to biomolecules in a way that facilitates data mining, designing of capture probes and primers, designing of microarrays, etc. The present invention also features methods for self-learning, self-engineering microarrays for the analysis of complex mixtures of nucleic acids and systems and apparatuses embodying such methods. In more particular aspects, the present invention features methods and systems that include inputting data into an applications program for execution on a computer, which applications program comprises a first mode that provides a training condition, and a second mode that provides a question and answer condition.

According to one aspect, the present invention features a method for improving the prediction of biological data. Such a method includes inputting data relating to one or more biomolecules into a universal database; identifying at least one of a plurality of groupings for data to be obtained (mined) from the universal database; inputting at least one of the groupings into a neural network program, which can analyze the data and generate new design rules for predicting biological data; and adapting a software program including initial design rules to the new design rules. In further embodiments, at least one of a plurality of groupings for data is identified for mining by comparing the inputted data with a predicted data resulting from the software program that includes initial design rules.

The groupings of data can be selected for mining when a discrepancy between the predicted biological data and the data relating to one or more biomolecules inputted into the universal database is observed. In addition, the data inputted into the universal database can be composed of a set of physical parameters, which characterize a particular biological molecule or molecules. Further, the experimental data relating to one or more biomolecules is preferably stored and grouped according to pre-set parameters in the universal database.

According to another aspect, the present invention features a computer-based system for prediction of biological data including a database for storing and retrieval of data relating to biomolecules, a design software program and a neural network program. The design software program includes initial design rules and the design software program is configured and arranged so as to be capable of adapting to new design rules. The neural network program analyzes data stored in the database and generates new design rules.

According to yet another aspect, the present invention features a computer related method for analysis of biological data. Such a method includes generating data from one or more biomolecules, inputting data into a universal database, and identifying at least one of a grouping of data to be mined from the universal database. The method also includes inputting at least one of the groupings into a design software program, the design software program comprising design rules (e.g. rules that establish parameters which are able to organize data such as parameters relating to oligonucleotide sequence design) and inputting an output of the design software program to a neural network program that analyzes the data and generates new design rules, which new rules are inputted into the design program.

According to a still further aspect, the present invention features a computer based system for analysis of data that includes an analysis substrate, a database, a design software program including design rules (e.g. rules that establish parameters which are able to organize data such as parameters relating to oligonucleotide sequence design), and a neural network program that analyzes data of the design software program. The analysis substrate preferably includes one or more biological molecules to be analyzed and/or data to be collected from.

According to another aspect, the present invention features an automated method for analysis of biological data. Such an automated method includes inputting data into a computer program which includes a first mode that provides a training condition, and a second mode that provides a question and answer condition.

According to a further aspect of the present invention, a database model is provided which may organize information relating to, e.g., sample preparation, expression analysis of experiment results, and intermediate and final results of mining gene expression measurements, gene sets, capture probe and primer design, etc. The data is downloaded into another software program which then allows for the analysis and design of capture probes, primers and the like. Information generated by this second software program is applied to a neural network program which generates new design rules that are loaded into the capture probe design program, thus setting up a learning loop. The model is readily translatable into database languages such as SQL and the like. The database model can scale to permit mining of information, collected from large numbers of samples.

In more specific embodiments of the present invention, the design rules can be used to predict the Tm of an oligonucleotide, for example. Various variables are included in rules for the prediction of Tm, such as the length of the oligonucleotide, the number of each type of the nucleobases, the position of nucleotides, the relative position of the DNA/RNA/LNA monomers (neighboring effect) and the like. Subsequently, an oligonucleotide can be actually prepared and hybridized to a complementing target. Data for the melting temperature Tm is generated, for example. The Tm data (actual Tm) together with the particulars of the oligonucleotide is inputted into a database and compared to the predicted Tm.

In a further specific embodiment, a score based on mathematical and statistical analysis is then calculated. Oligonucleotides performing as predicted receive one designation while oligonucleotides not performing as predicted receive another designation. Selected data from the database for oligonucleotides not performing as predicted is inputted into a neural network program. The neural network program can analyze the data and generate new static rules, which new rules can be inputted into the design program to improve or substitute the initial static rules.

In particularly specific embodiments, systems and methods of the present invention are used in conjunction with one or more analysis substrates that have loaded thereon one or more biomolecules such as peptides or oligonucleotides and information obtained from the analysis substrates are inputted into a database for analysis. In this way, the present invention yields a computer-based method for mining a plurality of experimental information. The method includes a variety of steps such as collecting information from experiments and chip designs. The method can include steps of selecting experiments to be mined.

In a yet another specific embodiment, the methodology of the present invention includes manipulating expression information. Such manipulating of expression information includes a variety of steps such as collecting information relating to results of experiments. Such manipulating also can include a step of gathering information about samples and information about the experiments, which can comprise an experimental analysis and the like. Further, the plurality of results of experiments can be transformed into a plurality of transformed information. Transformations can include normalizing, de-normalizing, aggregation, scaling, and the like. The methodology also can include steps of mining the plurality of transformed information and visualizing the plurality of transformed information.

In more specific embodiments, the methodology of the present invention also features a neural network self-organizing pattern classification system to properly classify input patterns of data that can be of many different forms. For example, such input pattern comprise capture probe or primer design data, prediction of melting temperatures, for use with or without a substrate analysis platform wherein each capture probe or primer or melting temperature represents a respective class.

In more specific embodiments, the neural network also can determine whether a correct class was selected to the input signal, which serves as a check and feedback mechanism for the system to learn new templates and to adjust its existing templates. When a correct classification is indicated by this system, the system does not simply accept this classification and proceed no further; rather it determines whether the classification has been stably classified. Preferably, if the selected class is too similar to another class (i.e. the template patterns of the different classes are too similar), then future classifications will not be stable and such future classifications could be classified erratically between the similar classes. If it is too similar, the system adjusts the templates to ensure their separation and stable classification. If an incorrect classification is made, the neural network system provides for the means for determining whether a template of a correct class exists.

Other aspects and embodiments of the invention are discussed below.

Definitions

The instant invention may be most clearly understood with reference to the following definitions:

As used herein, the term “analysis substrate”, “substrate platform”, “analysis platform”, “substrate analysis platform” or “slide element” or similar terms refers to the foundation upon which biomolecules may be immobilized, samples may be applied for analysis or biological assays may be carried out. Preferred analysis substrates can have an open configuration and generally confirm to the rectangular shape and thickness of a standard microscope slide, whether formed from a polymer or glass. For example, in the United States, typical slide elements have dimensions of 1 inch×3 inches. In Europe, typical slide dimensions include 25 mm×75 mm, or 26 mm×76 mm. Typical slide thicknesses are from about 1 mm to about 1.3 mm.

The term “biomolecule” as used herein is meant to indicate any type of nucleic acid, modified nucleic acid, protein, modified protein, peptide, modified peptide, small molecule, lectin, polysaccharide, hormone, drug, drug candidate, etc.

As used herein, a “target sequence” is any nucleic acid or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 8 to about 20. Preferably, longer sequence lengths can be used. However, it is well recognized that searches for commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

A computer readable medium shall be understood to mean any article of manufacture that contains data that can be read by a computer or a carrier wave signal carrying data that can be read by a computer. Such computer readable media includes but is not limited to magnetic media, such as a floppy disk, a flexible disk, a hard disk, reel-to-reel tape, cartridge tape, cassette tape or cards; optical media such as CD-ROM and writeable compact disc; magneto-optical media in disc, tape or card form; paper media, such as punched cards and paper tape; or on carrier wave signal received through a network, wireless network or modem, including radio-frequency signals and infrared signals.

### BRIEF DESCRIPTION OF THE DRAWINGS

For a fuller understanding of the nature and desired objects of the present invention, reference is made to the following detailed description taken in conjunction with the accompanying drawing figures wherein like reference character denote corresponding parts throughout the several views and wherein:

FIG. 1 is a schematic block diagram of an illustrative computer system on which the methodology of the present invention is implemented;

FIG. 2 is a high level flow diagram illustrating the methodology of the present invention;

FIG. 3 is another flow diagram illustrating the method in connection with a specific example; and

FIG. 4 is a schematic view illustrating an example for use of the present invention.

### DETAILED DESCRIPTION OF THE INVENTION

The present invention features methods for the analysis of biological data, more particularly as obtained from one or more biomolecules present on a microarray or other analysis substrate as well as systems, applications programs for execution on a computer and apparatuses embodying such methods.

Preferred methods of the invention for analysis of biological data include obtaining data such as from one ore more biomolecules that may be suitably present on a microarray or other analysis substrate. Data is generated from the biomolecules and that data is inputted into a database. A grouping of data (e.g. multiple data points that share a common characteristic whether a Tm value within a range of e.g. 1, 2, 3, 4, 5 or 10° C.; a highly conserved sequence (e.g. less than 1, 2, 3, 4 or 5 different nucleic acid or amino acid differences; and the like) is identified, and that grouped data is inputted into a software program that comprises design rules. An output from the program is obtained and inputted into a neural network program which can analyze the data and generate new design rules.

Preferably, the neural network inputs new rules to the design program thereby expanding the designing capabilities of the design software. The design rules may suitable comprise rules for designing nucleic acid capture probes, nucleic acid primers, and/or analysis substrates for biomolecules.

In a further preferred aspects, methods and systems are provided that comprise inputting data into a computer program which comprises a first mode that provides a training program, and a second mode that provides a question and answer condition. Preferably, the first mode is a training or learning condition and the second mode is a so-called oracle condition. The first training mode is suitably initially configured with an initial set of static rules contained in a design software program. Those rules can be directed to any of a variety of properties, e.g. any of a variety of properties of a biomolecule such as a sequence of a peptide or oligonucleotide, Tm of an oligonucleotide, etc. The second mode can serve as a question and answer condition.

In a further preferred aspect of the invention, methods and systems are provided that data generation from one or more biological molecules and use of a neural network to manipulate the data. Preferably, such methods and systems further a design software program to analysis the data, and the neural network provides input to the design software.

Analysis substrates used in methods and systems of the invention are suitably “loaded” with one or more biomolecules, i.e. a surface of the analysis substrate has one or more biomolecules thereon, preferably bound thereto. Biomolecule binding may be covalent, non-covalent, direct, indirect, via a linker, targeted, random, etc. Biomolecules may be attached through a single attachment to the surface of the substrate platform or via multiple attachments for a single biomolecule. Any type of binding method known to the skilled in the art may be used.

Nucleic acids that can be immobilized onto the substrate include RNA, mRNA, DNA, LNA, PNA, cDNA, oligonucleotides, primers, nucleic acid binding partners, etc. The nucleic acids for immobilization may be modified by any method known in the art. For example, the nucleic acids may contain one or more modified nucleotides, etc. and/or one or more modified internucleotide linkages, such as, phosphorothioate, etc. Particularly preferred 3′ and/or 5′ modifications include amino modifiers, thiols, and photoreactive ketones particularly quinones, especially anthraquinones.

Particularly preferred modified nucleic acids are those containing one or more nucleoside analogues of the locked nucleoside analogue (LNA) type as described in WO 99/14226, which is incorporated herein by reference. Additionally, the nucleic acids may be modified at either the 3′ and/or 5′ end by any type of modification known in the art. For example, either or both ends may be capped with a protecting group, attached to a flexible linking group, attached to a reactive group to aid in attachment to the substrate surface, etc.

As disclosed in WO 99/14226, LNA are a novel class of DNA analogues that form DNA- or RNA-heteroduplexes with exceptionally high thermal stability. LNA monomers include bicyclic compounds as shown immediately below:

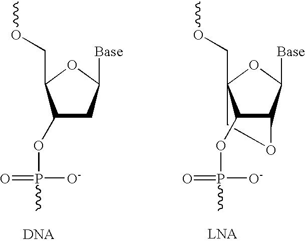

References herein to Locked Nucleoside Analogues, LNA or similar term refers to such compounds as disclosed in WO 99/14226.

Such oligonucleotides that contain modified residues (i.e. LNA or other non-natural residue) can be used in an analysis substrate that functions as an oligo array e.g. wherein a multitude of different oligos are affixed to a solid surface in a predetermined pattern (Nature Genetics, suppl. Vol. 21 Jan. 1999, 1-60 and WO 96/31557). The usefulness of such an array, which can be employed to simultaneously analyze a large number of target nucleic acids, depends to a large extent on the specificity of the individual oligos bound to the surface. The target nucleic acids may carry a detectable label or be detected by incubation with suitable detection probes.

Oligonucleotides containing LNA are easily synthesized by standard phosphoramidite chemistry. The flexibility of the phosphoramidite synthesis approach further facilitates the easy production of LNA oligos carrying all types of standard linkers, fluorophores and reporter groups.

Nucleic acids for immobilization onto the substrate may be either single stranded or double stranded and preferably contain from about 2 to about 1000 nucleotides, more preferably from about to 2 to about 100 nucleotides and most preferably from about 2 to about 30 nucleotides.

Polypeptides also can be immobilized onto the surface of the substrate platform. Particularly preferred polypeptides for immobilization are receptors, ligands, antibodies, antigens, enzymes, nucleic acid binding proteins, etc. Polypeptides may be modified in any way known to those skilled in the art. For example, polypeptides may contain one or more phosphorylations, glycosylations, etc. Additionally, polypeptides may be attached to a flexible linker and/or reactive to group to facilitate binding to the surface of the substrate.

Polypeptides for immobilization onto the substrate may be monomeric, dimeric or multimeric and preferably contain from about 2 to about 1000 amino acids, more preferably from about 2 to about 100 amino acids and most preferably from about 2 to about 20 amino acids. Polypeptides and nucleic acids for immobilization onto the substrate may be prepared separately and then applied onto the substrate surface. Methods for preparation of nucleic acids/oligos are known in the art, for example phosphoramidite chemistry.

Polypeptides and nucleic acids may be applied to the surface of the substrate by any method well known in the art. For example, polypeptides or nucleic acids may be manually pipetted onto the surface or applied using a robotics system. Preferably, polypeptides or nucleic acids are applied to the substrate using a micro spotting technique such as may be achieved with inkjet type technology.

Analysis substrates employed in the systems and methods of the invention also may be employed for relatively high density analysis, e.g. loaded for analysis with at least about 100 unique polypeptide sequences or nucleotides sequences per cm²of analysis area; or at least about 200, 300, 400, 500, 600, 700, 800 or 900 unique polypeptide sequences or nucleotides sequences per cm² of analysis area.

Biomolecules may be attached to the surface of the substrate using any method known in the art. Preferably biomolecules are attached to the surface using a photochemical linker which becomes active upon exposure to light of a defined wavelength. Most preferably biomolecules are attached to the surface using a quinone photolinker. Methods for photochemical immobilization of biomolecules using quinones are described in WO 96/31557, which is incorporated herein by reference.

Biomolecules may be attached directly to the analysis substrate surface or may be attached to the substrate through a flexible linker group. The linker group may be attached to the surface of the substrate before immobilization of the biomolecule or the linker group may be attached to the biomolecule before immobilization onto the substrate. For example, a nucleic acid may be modified with a linker group at either the 3′ or 5′ end prior to immobilization onto the substrate. Alternatively, an unmodified nucleic acid may be attached to the substrate which has been coated with linker groups. Similarly, a polypeptide may be modified with a group at either the amino terminus or carboxy terminus prior to immobilization onto the substrate. Alternatively, an unmodified polypeptide may be immobilized onto the substrate which has been coated with linker groups. The linker groups may be attached at any location within a nucleic acid or polypeptide chain but are preferably attached at either end of the polypeptide or amino acid chain. Linker groups for immobilization of biomolecules are well known in the art. Any linker group known in the art may be used for attachment of biomolecules.

Alternatively, polypeptides and nucleic acids may be synthesized in situ on the surface of the substrate. Methods for in situ synthesis of polypeptides and nucleic acids are well known in the art and include photolithographic techniques, protection/deprotection techniques, etc.

The analysis area of analysis substrates employed in systems and methods of the invention may be coated with a single biomolecule, with a random mixture of biomolecules or with a mixture of biomolecules wherein each unique biomolecule is located at a defined position so as to form an array. In a preferred embodiment the analysis area is coated with a library of polypeptides or nucleic acids wherein each unique nucleic acid or amino acid sequence is located at a defined location within the analysis area.

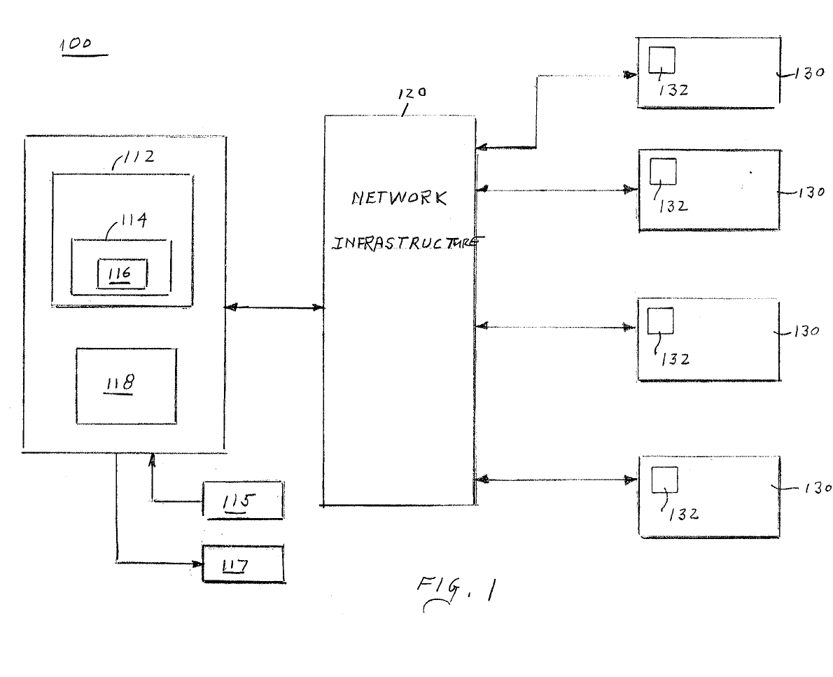

Referring now to the various figures of the drawing wherein like reference characters refer to like parts, there is shown in FIG. 1 a schematic block diagram of an illustrative computer system 100 including an applications program 116 for execution thereon which applications program includes functionalities and instructions and criteria that embody/implement the methodology of the present invention. In the illustrated embodiment, the computer system 100 includes a server 110, a network infrastructure 120 and one or more remote workstations 130. The illustrated embodiment, however, shall not be construed as limiting the invention to this particular form of a computer system as one skilled in the art will readily recognize that the application program and methodology described herein can be executed on any of a number of computer systems (e.g., a single PC) known in the art. In more particular embodiments, such a computer system or computer-based system for use in the present invention includes a central processing unit (CPU) or microprocessor, an input device(s) or mechanism(s), and output device(s) or mechanism, and a data storage device or mechanism.

The server 110 is provided to store the information and/or application program(s) of the present invention. It also is within the scope of the present invention for the server 110 to execute the applications program and carry out the functions described hereinafter and to communicate the results to the one or more remote workstations 130 requesting execution of the program. The server 110 includes a microprocessor or central processing unit (112) including memory (e.g., random access memory such as EDO, SDRAM, DDR) in which the applications program 116 is executed and a data storage device (e.g., magnetic high disk drive) on which is stored the informational database described hereinafter as well as the applications program and operating system.

The operating system is any one of the number of operating systems known to those skilled in the art that preferably includes a graphical user interface, such as for example, Windows 95™, Windows 98™ or Windows NT™ manufactured by Microsoft Corporation™, various flavors of the UNIX operating system, Macintosh operating systems, Lynx, OS/2, MVS, VM/CMS and the like. The applications program is implemented using any of a number of high level languages as is known to those skilled in the art including, but not limited to, Visual Basic, C, C++, Java, COBOL, Pascal and the like. In addition, database management systems as is known to those skilled in the art, such as Oracle, Sybase, Access, Progress and the like, are considered for use in implementing database management features of the present invention described hereinafter.

The server 110 is operably coupled to one or more input mechanisms or devices 115 as is known to those skilled in the art by which information, application programs, data and imagery (visual data) can be inputted into the server for processing and/or storage and is operably coupled to one or more output mechanisms or devices 117 as is known to those skilled in the art for displaying and/or providing a hard copy output. Such input devices 115 include, but are not limited to, keyboards, digital cameras, memory sticks, CD-RW or CD-ROM drives and/or DVD-ROM or DVD-RW drives. Such output devices 117 include, but are not limited to, CRT or LCD type of displays, printers, and/or data storage media such as magneto-optical disc's and/or removable magnetic storage disks (e.g., 3-½ in floppy disks).

The network infrastructure 120 is any of a number of infrastructures known to those skilled in the art that provides or establishes a communications link between the server 110 and each of the one or more remote workstations 130. In one embodiment, the network infrastructure 120 is implemented by providing a direct connection between the server 110 and a remote workstation 130 using telecommunications networks (e.g., by means of modems) or using the Internet. Alternatively, the network infrastructure comprises a local area network (LAN) or wide area network including for example token ring and Ethernet.

Each of the remote workstations 130 preferably includes a central processing unit (CPU) or microprocessor, an input device(s) or mechanism(s), and output device(s) or mechanism, and data storage device or mechanism. Each of the remote workstations 130 also includes an operating system and application programs for processing information, communicating between the server and the workstation. In particular embodiments, the remote workstation 130 also includes an application program embodying the methodology of the present invention. In the illustrated embodiment, each of the remote workstations 130 includes an output device 132, such as a display or printer, to provide the results in either visual or hard copy form to a particular user.

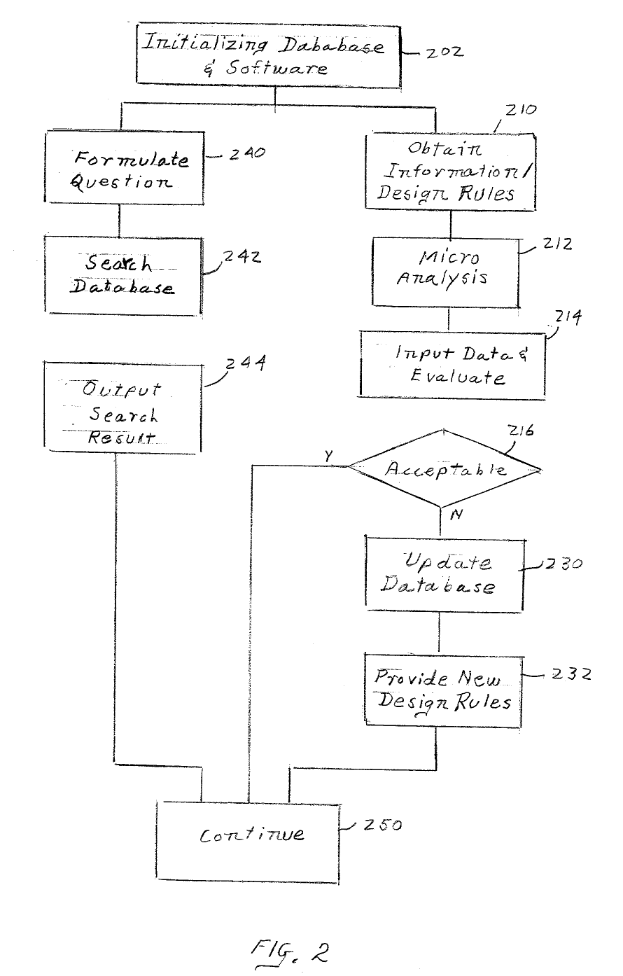

Now referring to FIG. 2, there is shown a high level flow diagram illustrating the methodology of the present invention as well as the functionalities, instructions and criteria of the applications program carrying out the methodology. In such a methodology, data is suitably generated from the biomolecules and that data is inputted into a database. A grouping of data is identified (e.g. multiple data points that share a common characteristic whether a Tm value with a range of 1, 2, 3, 4, 5, or 10° C.; a highly conserved sequence (e.g. less than 1, 2, 3, 4 or 5 different nucleic acid or amino acid differences; and the like), and that grouped data is inputted into a software applications program(s). An output from the program is obtained and inputted into a neural network program which can analyze the data. Additionally, data obtained from experiments, assays or from other analysis is inputted into the software or applications program(s) and inputted into a neural network program thereof, whereby the database and/or program can learn additional information.

As discussed hereinafter, such an applications program is preferably configured and arranged so as to operate in one of a first mode, a training mode or condition and a second mode, a question and answer mode or condition. Further, and as also discussed hereinafter, the described creation and the action of a neural network system takes place as an evolving process. The present invention is more particularly directed toward an automated system and method for defining research requirements, collecting data, analyzing data, visualizing data, building relationships between data items, and generating reports based on the data from a variety of sources. In addition to describing the methodology, the following describes the functionalities and the instructions and criteria of the applications program(s) of the present invention.

As an initial step, the methodology includes initializing the database, the “universal database” and initializing the software or applications program, step 202. Such initialization is further divided into an information manufacturing or experimental result stage, and a data storage and analysis stage. The term universal database as used herein refers to a database where all information obtained from experiments is or can be stored. Such a database, as further discussed hereinafter, can have certain design rules that allow the user to group data according to any desired criteria, e.g. rules that establish parameters which are able to organize data such as parameters relating to oligonucleotide sequence design.

Initially, and as part of the information manufacturing stage, generic training data is stored in the universal database. Such generic training data can include information the system requires for sequence recognition, clustering of groups of sequences, similarity of sequence recognition and/or target sequence recognition, or any other experimental information for example melting temperature (Tm) of an oligonucleotide. Thereafter portions of the data stored in universal database are selected for data analysis and for learning by the neural network of the applications program.

Once the generic training data is selected from the universal database, this data is applied to the neural network that initially has no intermediate nodes and no output nodes. The neural network then creates and modifies intermediate nodes and output nodes by learning with the generic training data. This learning process is similar to that used during the training mode or condition described hereinafter.

A neural network, for example, can include a plurality of input nodes for receiving the respective elements of the input vector. A copy of all of the elements of the input vector is sent to the next level of nodes in the neural network denoted as intermediate nodes. The intermediate nodes each encode a separate template pattern. They compare the actual input pattern with the template and generate a signal indicative of the difference between the input pattern and the template pattern. Each of the templates encoded in the intermediate nodes has a class associated with it. The difference calculated by the intermediate nodes is passed to an output node for each of the intermediate nodes at a given class. The output node then selects the minimum difference amongst the values sent from the intermediate nodes. This lowest difference for the class represented by the output node is then forwarded to a selector. The selector receives such values from each of the output nodes of all of the classes and then selects that to output value which is a minimum difference. The selector in turn, generates a signal indicative of the class of the intermediate node that sent the smallest difference value.

As used herein, “classes” refers to input data which is classified into groupings based on design rules. The design rules set up the parameters which are able to organize data and to classify input pattern signals of a given number of classes. The neural network program of the present invention can be programmed with parameters for the generation of specific design rules, for example, designing of capture probes for use in a selected micro-array experiment such as single nucleotide polymorphism (SNP) analysis. An example of a learning vector has been reported by Teuvo Kohonen, Gyorgy Barna, and Ronald Chrisley, “Statistical Pattern Recognition with Neural Networks: Benchmarking Studies”, Proceedings of IEEE International Conference on Neural Networks, Jul. 24-27, 1988, Vol. 1, pp. 61-68.

In general, when the data sets are installed in the information manufacturing stage, each particular character pattern is applied to a neural network that initially may have no intermediate nodes. The system then learns the character template that is being inputted and creates an appropriate intermediate node for that template. In this fashion, all or a substantial portion of the input is learned to create respective intermediate node templates. A private user may add new intermediate nodes or modifications of existing template patterns. For instance, if a user wishes to have a system that is already configured for using certain LNA capture probes for SNP analysis and wishes to add other capture probes such as RNA monomers, 2′-methyl monomers, monomers with modified bases, etc., the system learns to recognize these capture probes by applying the desired probes to the neural network. This application to the system causes the learning or creation of new templates for the desired capture probe patterns. These will be kept along with the already known LNA capture probes. Furthermore, the input data structures for any types of data character target may be made uniform so that a single system configuration may respond to all types of sequence recognition.

It is also an embodiment of the present invention to allow for manual tuning of data. More specifically, preferably, at this stage the data installed for the user system is modified or “re-tuned” at the user's private location. These privately tuned data are installed into the external memory of the user system. An example of the type of tuning that might occur is the creation of new array designs or loading of algorithms to sort out data according to need, such as inputting parameters for aligning certain sequences, preferably for the design of capture probes and primers.

After initialization, the process can proceed in either the first mode, the training mode, or the question and answer mode. The first training mode is suitably initially configured with an initial set of static rules contained in the applications or software program. Those rules can be directed to any of a variety of properties, more particularly any of a variety of properties of a biomolecule such as sequence of a peptide or oligonucleotide, Tm, as well as rules for designing nucleic acid capture probes, nucleic acid primers, and/or analysis substrates for biomolecules. As referred to herein, “design rules” are parameters which allow the respective software programs to analyze data based on selected criteria. In more particular embodiments, the design rules preferably are related to a configuration (sequence) of a oligonucleotide, e.g. having 3, 4, 5, 6, 7, 8, 9, 10, 15, 20, 30, 40, 50, 60, 70, 80, 90, 100 or more nucleotide residues, with those nucleotide residues suitably being “natural” DNA or RNA, or modified nucleic acids such as locked nucleic acids (LNA) e.g. as disclosed in WO 99/14226.

Under the training mode, a user obtains information or design rules relating to the particular work, experiment, etc. the user intends to perform, STEP 210. For instance, a user presents a question or other data input using the question and answer mode of the applications program, e.g. the user indicates the sequence of an oligonucleotide containing one or more LNA residues and the applications or software program returns a predicted Tm value. Alternatively, the user can form and inquiry concerning how to construct capture probes and primers for a given application such as for example a microarray slide experiment.

The microarray slide experiment is performed, step 212, and the results of this experiment are inputted into the universal database for analysis and evaluation, step 214. Although the methodology is described in connection with a micro-array slide experiment, this shall not be construed as particular limiting the methodology and system of the present invention to this particular application or use, for other suitably uses are contemplated and considered. More particularly, the experimental results are evaluated using the neural network of the applications program to determine their consistency or agreement with the predicted characteristic or parameter and/or the efficacy of the capture probe or primer in performing its function, step 216. If it is determined that the experimental results are not consistent/not acceptable (NO, step 216) then the application program updates the database, step 230, and provides the design rules and/or predicted characteristics or parameters based on the new experimental information. If it is determined that the experimental results are consistent/acceptable (YES, step 216) or after completion of the updating process of steps 230, 232 the process continues, step 250. In this way, a continuous learning or training loop is thereby formed by execution of the application program.

In another exemplary implementation, analysis information from a micro-array slide experiment is inputted into a universal database and the data is suitably stored until it is ready to be analyzed. The data is subsequently downloaded into the applications program using another database with basic design rules on how to construct capture probes and primers. In more particular embodiment, the applications program preferably includes initially a four dimensional matrix with four basic design rules on how to construct capture probes and primers. For example, genetic information from databases are downloaded into the design software program, and polymorphisms to be analyzed are highlighted. The applications or software program automatically designs the corresponding capture probes and primers based on the design rules contained in the instructions and criteria of the program.

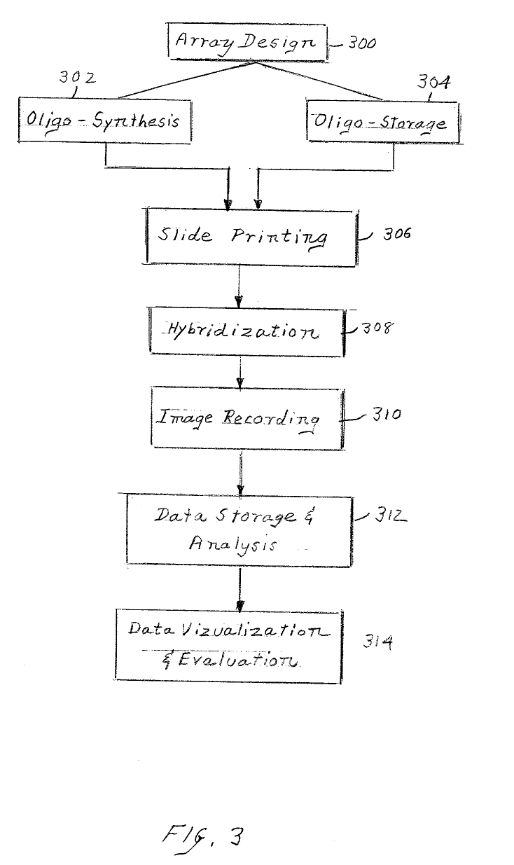

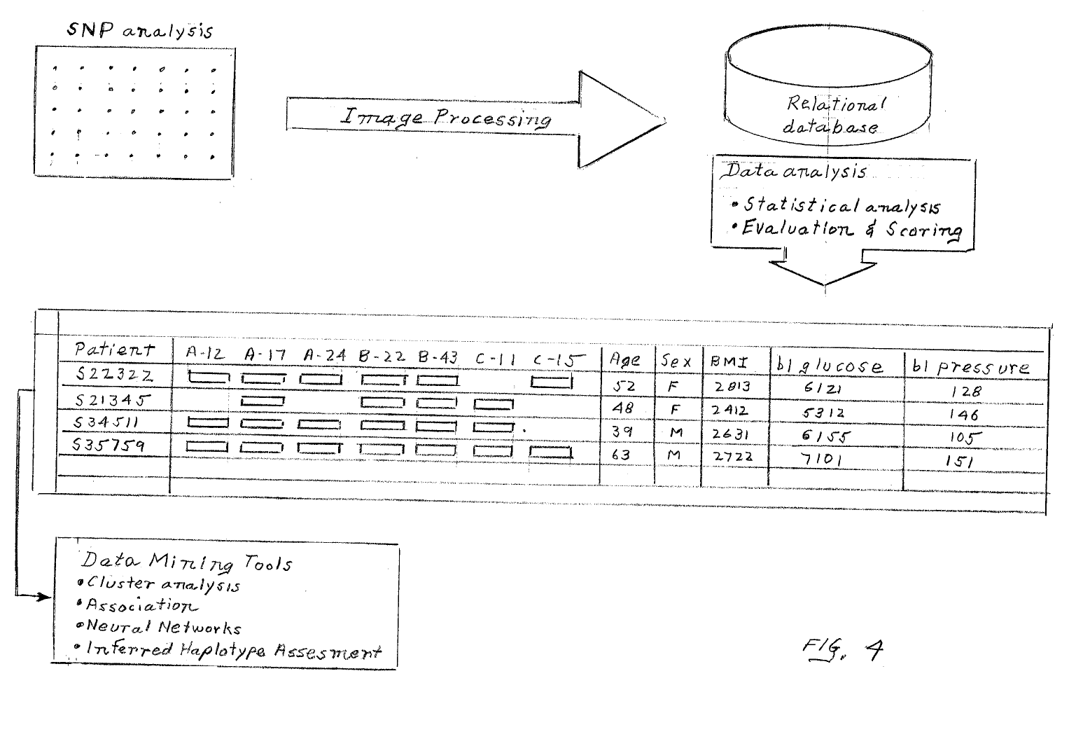

From the information generated by the applications program, arrays are designed, step 300, capture probes and primers are synthesized steps 302, 304, arrays are printed, step 306 and/or hybridization experiments performed, step 308, FIG. 3. Further images are recorded, step 310. After analysis all results are once again transferred to the universal database, and the analysis stage of experimental data is then suitably carried out by using, the neural network of the applications/ software program, steps 312, 314. The applications program preferably thereafter generates new design rules that are in turn loaded into the universal database/ applications program, thereby forming a continuous learning or training loop and thereby expanding the designing capabilities of the applications software program. As indicated above, these new design rules may suitably comprise rules for designing nucleic acid capture probes, nucleic acid primers, and/or analysis substrates for biomolecules. For illustrative purposes, the process illustrated in FIG. 3 assumes that the system is being utilized for microarray analysis, such as SNP analysis which is illustrated in FIG. 4.

In the foregoing reference is made to a database and applications program, however, it is within the scope of the present invention for the applications program to comprise one or more applications programs and the database to comprise one or more databases. In a particular embodiment of the present invention, the database is the commercially available database EURAYMatchMaker™ (available from Exiqon A/S of Vedbaek, Denmark), which holds all or a substantial portion of the information the system requires for sequence recognition, clustering of groups of sequences, similarity of sequence recognition and/or target sequence recognition, or any other experimental information. The applications program and database including the basic design rules on how to construct capture probes and primers is the commercially available program and database EURAYdesign™ (available from Exiqon A/S of Vedbaek, Denmark). The analysis stage of experimental data is carried out by using, a commercially available neural network (software) program, NEURAY™, (available from Exiqon A/S of Vedbaek, Denmark). NEURAY™ also generates new design rules which are loaded into EURAYdesign™, thereby forming the continuous learning or training loop.

In more specific embodiments, the design rules are directed to predict the Tm of an oligonucleotide. Various variables are included in rules for the prediction of Tm, such as the length of the oligonucleotide, the number of each type of the nucleobases, the position of nucleotides, the relative position of the DNA/RNA/LNA monomers (neighboring effect) and the like. Subsequently, the oligonucleotide is actually prepared and hybridized to a complementing target and data for the melting temperature Tm is generated. The analyzed oligonucleotide can be present in any of a variety of formats, e.g. on an analysis substrate or not attached to an analysis substrate.

An example of the working database is available from Exiqon A/S and through the Internet at www.LNA-tm.com and can be used for Tm measured in solution. The Tm data (actual Tm) together with the particulars of the oligonucleotide is inputted into a database and compared to the predicted Tm. In a preferred system, a score based on mathematical and statistical analysis is calculated. For example, in one system, oligonucleotides performing as expected receive a first value e.g. a score of 1 while oligonucleotides not performing as predicted receive a distinct value e.g. a score between 1 and 0. Selected data from the database for oligonucleotides not performing as predicted is inputted into a neural network of the applications program. The neural network of the applications program can analyze the data and generate new static rules, which new rules can be inputted into the design program to improve or substitute the initial static rules.

As used herein, “static rules” include those rules which are initially programmed into that portion of the applications program or software concerning the first mode which serve as a basis for setting up the parameters for analysis of data to generate an output. For example, these rules can be designed to predict the Tm of an oligonucleotide. The static rules suitably will include information regarding an oligonucleotide sequence such as the sequence, the number of nuclear bases, the types of nuclear bases, the percentage nuclear bases composition of an oligonucleotide, the Tm's of known duplex oligonucleotide sequences and the like.

As indicated above, the applications program of the present invention also preferably includes a second mode, a answer and question or oracle mode or condition. In the second mode, for example, a user of the system or method provides a question, or other input such as a physical property of a biological material such as the sequence of an oligonucleotide or polypeptide, hybridization characteristics of an oligonucleotide, and the like. The second mode then provides an output, e.g. a predicted Tm value based on a provided oligonucleotide sequence including a sequence that contains one or more locked nucleoside acid (LNA) residues or other modified nucleic acid residues responsive to the question or other input.

In the second mode, a user formulates the question or query for the desired information, property or the like and inputs this question into question and answer portion of the applications program, step 240. The application program using any of the number of search techniques known to the skilled in the art and appropriate for use with the universal database, causes the database to be searched, step 242. As indicated in the discussion above, the result of this search is outputted and provided to the user, step 244, and thereafter the process continues, step 250.

For example, the universal database and the database management techniques embodied in the applications program can be configured and arranged to as to be used with SQL database queries. The database management techniques being implemented can, for example, comprise a search engine and a database implementer (DBI) that are used to generate an optimal plan for an input query having specified required physical properties. The search engine generates a solution space from which an optimal plan is selected. The solution space is defined by a set of rules and search heuristics provided by the DBI. The rules are used to generate solutions and the search heuristics guide the search engine to produce more promising solutions rather than all solutions.

The database query can be, for example, represented as a query tree containing one or more expressions. An expression contains an operator having zero or more inputs that are expressions. The query optimizer can utilize two or more types of expressions, such as for example: logical expressions, each of which contain a logical operator; and physical expressions, each of which contain a physical operator specifying a particular implementation for a corresponding logical operator. An implementation rule transforms a logical expression into an equivalent physical expression and a transformation rule transforms a logical expression into an equivalent logical expression. The database query can be initially comprised of logical expressions. Through the application of one or more implementation and transformation rules, the logical expressions in the database query are transformed into physical expressions resulting in a solution.

In order to prevent the generation of redundant expressions, each rule can be classified as being context-free or context-sensitive. A context-free rule is applied once to an expression, while a context-sensitive rule is applied once to an expression for each optimization goal.

A search data structure is used to store the expressions that are generated during the search process including those that are eliminated from consideration. The search data structure can be organized into equivalence classes denoted as groups. Each group represents expressions that are equivalent to one another. Equivalence in this sense denotes those expressions that contain semantically equivalent operators, have similar inputs, and require the same characteristic inputs and produce the same characteristic outputs (otherwise referred to as group attributes). The set of characteristic inputs represent the minimum number of values required for the expression's operator and for any input expressions associated with it. The set of characteristic outputs represent the minimum number of values that the expression supplies to any parent expression associated with the expression.

Each group can include one or more logical expressions, zero or more physical expressions, zero or more design plans, and zero or more design contexts. The expressions contained in a group are semantically equivalent to one another. A context is associated with an optimization goal and contains reference to (potentially) one optimal solution (plan) and other candidate design plans. By explicitly distinguishing between design plans and physical expressions, multiple design plans can be generated from the same physical expression given different required physical properties.

Initially the group attributes for each logical expression of the input query are determined and used to store each expression in an appropriate group in the search data structure. For example, melting temperatures for desired oligonucleotides or SNP analysis of various patient haplotypes. As the optimizer applies rules to the logical expressions, additional equivalent expressions, plans and groups are added. The group attributes of the newly generated expressions are computed in order to determine whether a duplicate of the newly generated expression is stored in the search data structure. A duplicate expression is one that has the same operator, number of inputs, ordinality of inputs, and group attributes, for example. Duplicate expressions are not inserted into the search data structure.

The search engine can utilize a search procedure to generate a solution by partitioning the database query into one or more subproblems where each subproblem can contain one or more expressions. Some of the subproblems form a subtree having other subproblems as inputs. Each subproblem has an associated set of required physical properties that satisfies the constraints imposed by its associated parent subproblem's required physical properties. A solution to each subproblem is generated in accordance with an order that generates a solution for each input subproblem before a solution for its associated parent subproblem is generated. The solution for the database query is then obtained as the combination of the solutions for each of the subproblems. The search procedure can utilize a branch and bound technique for generating solutions for each subproblem.

The present invention may apply one or more pruning heuristics to the data to be analyzed to prevent unnecessary application and implementation of rules. Such pruning heuristics can significantly reduce the search space by eliminating nodes before optimizing the expression. For example, certain haplotypes such as haplotype A and B from a selected patient pool may be selected from the database.

Prior to applying a rule to an expression, one embodiment of the present invention identifies all possible bindings that match a rule's pattern. The purpose of a binding is to find all possible expressions that can match a rule's pattern in order to generate every possible equivalent expression. An expression in the search data structure is stored with pointers representing each input, if any. Each pointer has a link mode that allows it to reference the group associated with the input or a particular input expression in the group, for example. When a pointer's link mode is in memo mode, each pointer addresses the group of each input. When the link mode is in binding mode, each pointer addresses a particular expression in the input's group.

In binding an expression that has inputs, the link mode of the expression is set to binding mode. The input pointers of the expression are set to address a particular input expression stored in the associated group, thus forming a specific subtree. Further subtrees or bindings are formed by incrementing the input pointers appropriately to form other subtrees representing a different combination of the input expressions.

Prior to applying a design rule to an expression the present invention can also identify the complexity of the query. If the complexity of the query is above a threshold, the present invention determines whether the rule should be applied based upon several factors including the type of rule and the position of the node in the search space. Those rules that need not be applied are randomly pruned. Pruned rules are not applied, while those rules that are not pruned are applied.

The DBI can be comprised of search heuristics in the form of guidance methods that select a set of rules for use in generating a plan for each subproblem. A guidance method, e.g., OnceGuidance, can be used to prevent the needless generation of redundant expressions that result from the subsequent application of a rule to the rule's substitute. The OnceGuidance guidance method can be used in certain cases to select a set of rules that are applicable to a rule's substitute without including those rules that will regenerate the original expression. Thus different analysis, capture probes and primers may be designed according to new design rules.

The search engine utilizes a series of tasks to implement the search procedure. Each task performs a number of predefined operations and schedules one or more additional tasks to continue the search process if needed. Each task terminates once having completed its assigned operations. A task stack can be used to store tasks awaiting execution. The task stack is preferably operated in a last-in-first-out manner. A task scheduler is used to pop tasks off the top of the stack and to schedule tasks for execution.

A garbage collection task can be scheduled whenever two groups of the search data structure are merged. The merger of two groups occurs as a result of the application of certain rules, such as the “elimination group by” rule. In this case, the elements of one group are merged with the elements of a second group when its determined that the two groups share a common expression. The first group obtains the group identifier of the second group. The garbage collection task is then scheduled to update any references to the first group by any of the expressions in the search data structure and to eliminate any duplicate expressions in the merged groups.

In one application of the invention, a nucleotide sequence with a known Tm is recorded on computer readable media. A skilled artisan can readily appreciate how any of the presently known computer readable mediums can be used to create a manufacture comprising computer readable medium having recorded thereon a nucleotide sequence of the present invention. As, used herein, “recorded” refers to a process for storing information on computer readable medium. A skilled artisan can readily adopt any of the presently known methods for recording information on computer readable medium.

A variety of data storage structures are available to a skilled artisan for creating a computer readable medium having recorded thereon, for example, Tm data from nucleotide sequences from known sources, unknown sources, synthetic oligomers wherein the Tm would be important for designing primers in PCR, nucleotide sequencing and the like. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of data processor structuring formats (e.g. text file or database) in order to obtain computer readable medium having recorded thereon the relevant information. Computer software also is publicly available which allows a skilled artisan to access sequence information provided in a computer readable medium. For example, software which implements the BLAST (Altschul et al., J. Mol. Biol. 215:403-410 (1990)) and BLAZE (Brutlag et al., Comp. Chem. 17:203-207 (1993)) search algorithms on a Sybase system is used to identify open reading frames (ORFs) within a nucleic acid sequence.

As used herein, “search means” refers to one or more programs or code segments comprising the applications program of the present invention, which are implemented to compare a target sequence or target structural motif with the sequence information stored within the data storage means. Search means are used to identify fragments or regions of a known sequence which match a particular target sequence or target motif. A variety of known algorithms are disclosed publicly and a variety of commercially available software for conducting search means are and can be used in the computer-based systems of the present invention. Examples of such software includes, but is not limited to, MacPattern (EMBL), BLASTN and BLASTA (NPOLYPEPTIDEIA). A skilled artisan can readily recognize that any one of the available algorithms or implementing software packages for conducting homology searches can be adapted for use in the present computer-based systems.

Analysis substrates used in methods and systems of the invention may include monomer nucleotide sequences such as described herein. When a sample is applied to the analysis platform, the nucleotides may or may not bind to the probes. The nucleotides have been tagged with fluorescein labels to determine which probes have bonded to nucleotide sequences from the sample. The prepared samples are placed in a scanning system. Such a scanning system includes for example, a detection device such as a confocal microscope or CCD (charge-coupled device) that is used to detect the location where labeled receptors have bound to the substrate. The output of the scanning system is an image file(s) indicating, in the case of fluorescein labeled receptor, the fluorescence intensity (photon counts or other related measurements, such as voltage) as a function of position on the substrate. Since higher photon counts will be observed where the labeled receptor has bound more strongly to the array of polymers, and since the monomer sequence of the polymers on the substrate is known as a function of position, it becomes possible to determine the sequence(s) of polymer(s) on the substrate that are complementary to the receptor.

Associated with samples are attributes. Some of the attributes are strings or values identifying concentrations, sample preparation dates, expiration dates, and the like. Other attributes identify characteristics that are highly useful in searching for genes or ESTs of interest such as the disease state of tissue, the organ, or species from which a sample is extracted. A sample may have more than one attribute, and an attribute can describe more than one sample item. Examples of attribute types are “concentration,” “preparation date,” “expiration date,” etc. Another example of an attribute type would be “specimen type” where possible values would correspond to “tissue,” “organ culture,” “purified cells,” “primary cell culture,” “established cell line,” and the like. Another example might be “ethnic group” where different values may correspond to “East Asian,” “Native American,” for example.

The image files and the design of the chips are inputted to the universal database, such as EURAYMatchMaker™. This database can, for example, align base sequences, or determine expression levels of e.g. genes or expressed sequence tags, predict Tm values, etc. The expression level of a gene or EST is herein understood to be the concentration within a sample of mRNA or protein that would result from the transcription of the gene or EST. The universal database such as EURAYMatchMaker™ also maintains information used to analyze expression and the results of expression analysis. Contents of expression analysis may include tables listing analyses performed, analysis results, experiments performed, sample preparation protocols and parameters of these protocols, substrate analysis platform designs, capture probe and primer designs etc.

To facilitate investigations of this kind the universal data base such as EURAYMatchMaker™, provides for a mining database. The mining database may include duplicate representations of data obtained during experimentation. The universal database such as EURAYMatchMaker™ may also include various tables to facilitate mining operations conducted by a user who operates a querying and mining system. Querying and mining system includes a user interface that permits an operator to make queries to investigate expression of genes, ESTs, capture probe and primer designs. Selected information from the universal database such as EURAYMatchMaker™ can be downloaded into the applications program such as the EURAYdesign™ software program portion thereof.

The following glossary of terms used in connection with queries or searches are provided as follows, however, the following glossary is not exhaustive and shall not to be used to limit the scope of the present invention.

Relational Expression: A relational expression is one that produces a table as its output, such as a join or scan. Relational expressions differ from value expressions that contain arithmetic operators and produce a value as an output. A relational expression can be a physical expression or a logical expression or both.

Logical Expression: A logical expression contains a logical operator of a certain arity (having a required number of inputs) and whose inputs are logical expressions. The arity of the logical operator is >=0. The inputs are also referred to as children or input expressions.

Physical Expression: A physical expression includes a physical operator of a certain arity and whose inputs are physical expressions. Similarly, the arity of the physical operator is >=0. The inputs are also referred to as children or input expressions.

Table Expression: A table expression is a relational expression that produces a table or set of rows. Examples of table expression operators include Scan (or Retrieve), Join, Union, and Group By.

Logical Operator: A logical operator represents an implementation-independent operation (e.g., join or scan).

Physical Operator: A physical operator specifies a particular implementation method or procedure (e.g., hashjoin, mergejoin, etc.).

Expression tree: An expression tree corresponds to a relational expression having one or more logical or physical expressions. The expression tree includes one or more nodes, each node is classified as a logical expression or a physical expression. Each node can contain zero or more inputs, each input being a relational expression. The expression tree includes one or more levels, each level containing nodes that are inputs to a node of a preceding level. The root node represents a relational expression having the top-most operator and positioned in the first level.

Left Linear Tree: A left linear tree is an expression tree, where the right (inner) child of every join node in the tree is a scan or aggregate on a single table in the query.

ZigZag Tree: A zigzag tree is a generalization of linear tree where the single table child can be either the inner or outer child of the join node. For example, a zigzag tree may be an expression tree where at least one of children of every join node in the tree is a scan or aggregate on single table in the query.

Bushy Tree: a bushy tree is an expression tree with no restriction on the number of tables involved in any join input subtree.

Plan: A plan is an expression tree that is comprised solely of physical expressions. A plan is associated with a particular optimization goal and is considered complete when an associated cost and required physical properties is assigned to it. The term plan and solution are used in this document interchangeably.

Query tree: A query tree is an expression tree that corresponds to the input query that is to be optimized. Also, query tree is used to refer to the most optimum design rules used in the designing of capture probes and primers. The query tree contains one or more nested logical expressions.

Optimization rule: An optimization rule defines how the optimizer is to transform the input query into other semantically equivalent forms.

Transformation rule: A transformation rule transforms a logical expression into a semantically equivalent logical expression (e.g., SNP analysis of selected genes and design of new capture probes and primers based on the analysis).

Implementation rule: An implementation rule transforms a logical expression into a semantically equivalent physical expression by substituting one or more logical operators in the logical expression with physical operators (e.g., join may be implemented by mergejoin). The repeated application of implementation rules results in a plan that is comprised only of physical expressions.

Pattern and Substitute: An optimization rule includes a pattern and a substitute, both of which are expression trees. The pattern is the “before” expression that is matched with the expression that is being optimized. The substitute represents the semantically equivalent expression that is generated by applying the rule. A rule's pattern matches an expression when the expression contains the same operators in the same position as the rule's pattern.

Cut operator: A cut operator is an input to a rule's pattern that can be matched to any operator.

Tree operator: A tree operator is an input to a rule's pattern that is matched to an entire expression tree.

Memo: A memo is a search data structure used by the optimizer for representing elements of the search space. The Memo is organized into equivalence classes denoted as groups. Each group includes one or more logical and physical expressions that are semantically equivalent to one another. Expressions are semantically equivalent if they produce the identical output. Initially each logical expression of the input query tree is represented as a separate group in Memo. As the optimizer applies rules to the expressions in the groups, additional equivalent expressions and groups are added. Each croup also contains one or more plans and contexts. A context represents plans having the same optimization goal.

Physical properties: A physical property specifies the manner for representing the output of an expression.

Optimization goal: An optimization goal represents the required physical properties or design rules that are associated with certain experiments. For example, in genotyping. One embodiment of the present invention operates in the context of a system, described below, for analyzing biological or other materials using arrays that themselves include capture probes or primers that may be made of biological materials such as LNA monomers, RNA monomers, DNA monomers, 2′-methyl monomers, oxy-LNA monomers and any other monomers with modified bases.

As indicated above, although a network system is illustrated in FIG. 1, an independent computer for each system can perform the computer-implemented functions of these systems or one computer can combine the computerized functions of two or more systems. Many other devices or subsystems may be connected in a similar manner.

All documents mentioned herein are incorporated herein by reference in their entirety.

The invention has been described in detail with reference to preferred embodiments thereof. However, it will be appreciated that those skilled in the art, upon consideration of this disclosure, may make modifications and improvements within the spirit and scope of the invention.

What is claimed is:

1. A method for improving the prediction of biological data, comprising:
inputting data relating to one or more biomolecules into a universal database;
identifying at least one grouping of data to be obtained from the universal database;
inputting at least one of the data grouping into a neural network program, which can analyze the data and generate new design rules for predicting biological data; and
adapting a software program including initial design rules to the new design rules.

2. The method of claim 1 wherein the at least one of a plurality of groupings for data is identified for obtaining by comparing the inputted data with a predicted data resulting form the software program including initial design rules.

3. The method of claims 1 or 2 wherein a grouping of data is selected for obtaining from the database when a discrepancy between the predicted biological data and the data relating to one or more biomolecules inputted into the database is observed.

4. The method of any one of claims 1 to 3 wherein the data inputted into the universal database is composed of a set of physical parameters, which characterize a particular biological molecule.

5. The method of any of the claims 1 to 4 wherein experimental data relating to one or more biomolecules is stored and grouped according to pre-set parameters in the universal database.

6. The method of claim 5 wherein the data is grouped according to species, sequence homologies, and/or particular molecules.

7. The method of any one of claims 1 through 6 wherein the software program comprises at least a four dimensional matrix with design rules on how to construct capture probes and primers.

8. The method of claim 7 wherein new design rules can be implemented allowing for an expansion of rules.

9. The method of any one of claims 1 through 8 wherein data obtained from peptide or nucleic acid sequences are inputted into the neural network program which designs new rules based upon inputted data.

10. The method of claim 9 wherein the new design rules are inputted into the design software program, thereby forming a training loop.

11. The method of any one of claims 1 to 10 wherein the one or more biomolecules are present on an analysis substrate.

12. The method of any one of claims 1 to 12 wherein the one or more biomolecules are peptides or nucleic acid compounds.

13. The method of any one of claims 1 to 12 wherein the biomolecule is an oligonucleotide.

14. The method of claim 13 wherein the oligonucleotide comprises natural nucleotides.

15. The method of claim 14 wherein the oligonucleotide further comprises non-natural nucleotides.

16. The method of any one of claims 1 through 15 wherein the design software program comprises rules for designing nucleic acid capture probes or nucleic acid primers.

17. A computer based system for prediction of biological data comprising:
a database for storing and retrieval of data relating to biomolecules;
a design software program comprising initial design rules, the design software program being configured to be capable of adapting to new design rules; and
a neural network program, which can analyze data stored in the database and generate new design rules.

18. The system according to claim 17 wherein the neural network program can input new design rules to the design software program.

19. The system of claims 17 or 18 wherein the design software program comprises rules for designing nucleic acid capture probes or nucleic acid primers.

20. A computer related method for analysis of biological data, the method comprising:
generating data from one or more biomolecules;
inputting data into a universal database;
identifying at least one grouping of data to be mined from the universal database;
inputting at least one data grouping into a design software program, the design software program comprising of design rules;
inputting an output of the design software program to a neural network program which can analyze the data and generate new design rules, which new rules can be inputted into the design program.

21. The method of claim 20 wherein the one or more biomolecules are present on an analysis substrate.

22. The method of claim 20 or 21 wherein the neural network program inputs new rules to the software program thereby expanding the designing capabilities of the software program.

23. The method of any one of claims 20 through 22 wherein the design rules comprise rules for designing nucleic acid capture probes, nucleic acid primers, and/or analysis substrates for biological materials.

24. A computer based method comprising generating data from one or more biological molecules and utilizing a neural network to manipulate the data.

25. The method of claim 24 further comprising utilizing a design software program to analysis the data.

26. The method of claim 24 or 25 wherein the neural network provides input to the design software.

27. The method of any one of claims 20 through 26 wherein the biological molecules are on a surface of the analysis substrate.

28. The method of claim 27 wherein the biological molecules comprise a plurality of nucleic acid sequences immobilized on the substrate surface.

29. The method of any one of claims 20 through 28 wherein the biological molecules comprise modified nucleic acids.

30. The method of any one of claims 20 through 29 wherein the biological molecules comprises one or more locked nucleic acids.

31. The method of any one of claims 20 through 30 wherein the biological molecules comprise nucleic acid sequences that contain at least one phosphorothioate internucleoside linkage.

32. The method of any one of claims 20 through 30 wherein the biological molecules are nucleic acid sequences that have each internucleoside linkage being a phosphorothioate linkage.

33. The method of any one of claims 20 through 32 wherein the biological molecules comprise nucleic acid sequences that comprise at least one modified nucleotide and at least one modified internucleoside linkage.

34. The method of any one of claims 20 through 32 wherein experimental data obtained from the substrate analysis platform is stored and grouped according to pre-set parameters.

35. The method of claim 34 wherein the data is grouped according to species, sequence homologies, and/or particular molecules.

36. The method of any one of claims 20 through 35 wherein the design software program comprises at least a four dimensional matrix with basic design rules on how to construct capture probes and primers.

37. The method of claim 36 wherein new design rules can be implemented allowing for an expansion of rules.

38. The method of any one of claims 20 through 37 wherein data obtained from peptide or nucleic acid sequences are inputted into the neural network program which designs new rules based upon inputted data.

39. The method of claim 38 wherein the new design rules are inputted into the design software program, thereby forming a training loop.

40. A computer based system for analysis of data comprising:
an analysis substrate;
a database;
a design software program comprising design rules; and
a neural network program which can analyze data of the design software program.

41. The system of claim 40 wherein the analysis substrate comprises one or more biological molecules.

42. The system of claim 40 wherein the analysis substrate on a surface thereof comprises one or more peptide or nucleic acid sequences.

43. The system of any one of claims 40 through 42 wherein the neural network program can input new rules to the design program.

44. The system of any one of claims 40 through 43 wherein the design rules comprise rules for designing nucleic acid capture probes, nucleic acid primers, and/or substrate analysis platform arrays for biological materials including nucleic acid and peptides.

45. An automated method for analysis of biological data, the method comprising inputting data into a computer program which comprises a first mode that provides a training condition, and a second mode that provides a question and answer condition.

46. The method of claim 45 wherein the first mode comprises an initial set of static rules used in the analysis and prediction of biological data.

47. The method of claim 45 or 46 wherein the first mode comprises rules with variable parameters.

48. The method of any one of claims 45 through 47 wherein the variables are comprised of oligonucleotide lengths, number and type of nucleobases in said oligonucleotides, positions of nucleotide bases in said oligonucleotides, and positions of DNA/RNA/LNA monomers.

49. The method of any one of claims 45 through 48 wherein an oligonucleotide is prepared and hybridized to a complementing target and data for the melting temperature (Tm) is generated.

50. The method of claim 49 wherein a measured Tm of a particular oligonucleotide is inputted into a database.

51. The method of claim 49 or 50 wherein a measured Tm of an oligonucleotide is compared to a predicted Tm of the oligonucleotide.

52. The method of any one of claims 44 through 50 wherein a score based on mathematical and statistical analysis is calculated.

53. The method of claim 52 wherein oligonucleotides performing as predicted receive a first designation while oligonucleotides not performing as predicted receive a second designation distinct from the first designation.

54. The method of any one of claims 45 through 53 wherein data from the database for oligonucleotides not performing as predicted is inputted into a neural network program.

55. The method of claim 54 wherein the neural network program analyses data and generates new static rules, which new rules can be inputted into the design program to improve or substitute the initial static rules.

56. The method of any one of claims 45 through 55 wherein the second mode is performed after the first mode.

57. The method of any one of claims 45 through 56 wherein the second mode comprises inputting data for which a predicted result is desired and a software program returns predicted data.
